(19) United States
(12) Patent Application Publication
(10) Pub. No.: US 2013/0045203 A1
(43) Pub. Date: Feb. 21, 2013

(54) Uses of Noscapine and Derivatives in Subjects Diagnosed with FAP

(71) Applicants: Harish C. Joshi, Decatur, GA (US); Vincent Yang, East Setauket, NY (US)

(72) Inventors: Harish C. Joshi, Decatur, GA (US); Vincent Yang, East Setauket, NY (US)

(73) Assignee: EMORY UNIVERSITY, Atlanta, GA (US)

(21) Appl. No.: 13/581,377

(22) PCT Filed: Feb. 25, 2011
(86) PCT No.: PCT/US11/26218

§ 371 Date: Oct. 29, 2012

Related U.S. Application Data
(60) Provisional application No. 61/309,482, filed on Mar. 2, 2010.

(51) Int. Cl. A61K 31/4355; A61K 33/24; A61K 31/513; A61K 31/7068; A61K 31/519; A61K 39/395; A61K 31/713; A61K 31/704; A61K 38/14; A61K 31/69; A61K 39/00; A61K 31/675; A61K 33/06; A61P 35/00; A61K 31/616

(52) U.S. Cl. 424/133.1; 514/291; 514/165; 424/649; 514/274; 514/49; 514/249; 424/142.1; 514/44 A; 514/34; 514/19.3; 514/64; 424/277.1; 514/89; 424/682

(57) ABSTRACT

This disclosure relates to methods of treating or preventing cancer comprising administering a pharmaceutical composition comprising noscapine or noscapine derivatives to a subject diagnosed with a mutated adenomatous polyposis coli (APC) gene.

This application claims priority to U.S. Provisional Application No. 61/309,482 filed 2 Mar. 2010, hereby incorporated by reference.

### BACKGROUND

Colorectal cancer, also called colon cancer or large bowel cancer or “CRC”, includes cancerous growths in the colon, rectum and appendix. Colorectal cancers arise from adenomatous polyps in the colon. These mushroom-shaped growths are usually benign, but some develop into cancer over time. Localized colon cancer is usually diagnosed through colonoscopy. On the cellular and molecular level, colorectal cancer starts with a mutation to the Wnt signaling pathway. When Wnt binds to a receptor on the cell, a chain of molecular events is set in motion ending with β-catenin moving into the nucleus and activating a gene on DNA. In colorectal cancer, genes along this chain are typically damaged.

Familial adenomatous polyposis (FAP), a hereditary condition that predisposes affected people to colon cancer, is caused by a mutated adenomatous polyposis coli (APC) gene. FAP patients develop hundreds to thousands of colonic polyps around their teenage years. Without treatment, the disease progresses to colorectal cancer. The NSAID Sulindac has been shown to cause regression of polyps in FAP patients. However, the drug is not effective as a sole treatment for FAP because drug resistance occurs, and cancer develops. Instead, the primary treatment for FAP remains the surgical removal of the colon. Thus, there remains a need to identify therapeutic treatments that treat or prevent colorectal cancer.

A natural alkaloid, noscapine, can bind tubulin with a 1:1 stoichiometry and reduce the transition of microtubule (MT) dynamics from growing to shortening phases and vice versa. Furthermore, a synthetic bromo-derivative of noscapine, 9-bromonoscapine (EM011), binds tubin with a higher affinity than noscapine without changing total microtubule polymer mass. See Aneja et al., Biochem Pharmacol, 2006,72:415-26 and Zhou et al., Mol Pharmacol, 2003, 63:799-807.

### SUMMARY

This disclosure relates to methods of treating or preventing cancer comprising administering a pharmaceutical composition comprising noscapine or noscapine derivatives to a subject diagnosed with a mutated adenomatous polyposis coli (APC) gene. In certain embodiments, the disclosure relates to the use of noscapine derivatives disclosed herein in the production of a medicament for the treatment or prevention of developing colon cancer.

In some embodiments, the noscapine derivative is a compound comprising Formula A:

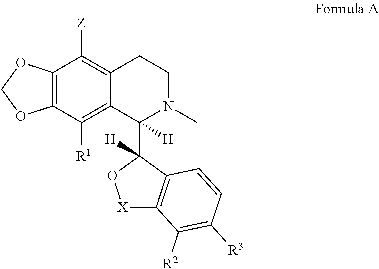

or pharmaceutically acceptable salts, prodrugs or derivatives thereof.

wherein Z is a halogen, nitro, or nitrogen wherein nitrogen may be optionally substituted with R⁴;

X is carbonyl (C═O) or methylene (CH₂) optionally substituted with R⁴;

R¹, R², and R³ are each independently an alkoxy optionally substituted with one or more R⁴; R⁴ is independently selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with R⁵;

R⁵ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl. In a typical embodiment, R¹, R², and R³ are each methoxy.

In certain embodiments, Z is hydrogen, X is methylene (CH₂) or a carbonyl (C═O) group optionally substituted with R⁴; R¹, R², and R³ are each independently an alkoxy optionally substituted with one or more R⁴.

Noscapine is 3-(4-methoxy-6-methyl-5,6,7,8-tetrahydro-[1,3]dioxolo[4,5-g]isoquinolin-5-yl)-6,7-dimethoxyisobenzofuran-1(3H)-one and a typical noscapine derivative is 3-(9-halo-4-methoxy-6-methyl-5,6,7,8-tetrahydro-[1,3]dioxolo[4,5-g]isoquinolin-5-yl)-6,7-dimethoxyisobenzofuran-1(3H)-one such as 3-(9-bromoe-4-methoxy-6-methyl-5,6,7,8-tetrahydro-[1,3]dioxolo[4,5-g]isoquinolin-5-yl)-6,7-dimethoxyisobenzofuran-1(3H)-one or 3-(9-chloro-4-methoxy-6-methyl-5,6,7,8-tetrahydro-[1,3]dioxolo[4,5-g]isoquinolin-5-yl)-6,7-dimethoxyisobenzofuran-1(3H)-one.

In certain embodiments, the subject is diagnoses with colon cancer or the subject is not diagnosed with colon cancer but is at risk of developing colon cancer. In certain embodiments, the subject is diagnosed with colonic polyps. In certain embodiments, the subject is less than 10, 11, 12, 13, 14, 15, 16, 17, or 18 years old.

In certain embodiments, the pharmaceutical compositions are administered in combination with a non-steroidal anti-inflammatory agent such as aspirin or sulindac or in combination with one or more other chemotherapeutic agents such as docetaxel, cis-platin, 5-fluorouracil, tegafur-uracil, capecitabine, leucovorin, oxaliplatin, irinotecan, panitumumab, oblimersen, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib, anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, gefitinib, erlotinib, dasatinib, imatinib, bevacizumab, combretastatin, thalidomide, and lenalidomide.

In certain embodiments, the pharmaceutical composition is administered in combination with cimetidine, vitamin B6, or calcium. In certain embodiments, the pharmaceutical composition is administered in combination with a cancer vaccine such as a pox virus vector that expresses a tumor-associated antigen, 5T4.

In certain embodiments, the subject may be diagnosed with a tumor confined within the wall of the colon.

In certain embodiments, the pharmaceutical composition is administration before or after the subject undergoes surgical removal of the colon or before or after the subject undergoes radiation therapy.

### BRIEF DESCRIPTION OF THE FIGURES

FIG. 1A illustrates 9-bromonoscapine (EM011) and shows data on viable and dead cell-count obtained using the trypan blue exclusion assay.

FIG. 1B I shows FACS analysis for wild type MEFs.

FIG. 1B II shows FACS analysis for Apc^{Min/-} MEFs.

FIG. 1B III shows data quantified for both WT and Apc^{Min/+} MEFs.

FIG. 1C shows confocal immunofluorescence analysis of the WT and Apc^{Min/+} MEFs

FIG. 1D shows data on the FACS analysis for wild type MEFs and Apc ^{Min/+} MEFs after EM011 treatments.

FIG. 2A illustrates a hypothesis as to why EM011 treatment lowers β-catenin levels and activity in Apc^{Min/+} MEFs.

FIG. 2B shows data of quantitative western blot measurements of lowered levels of β-calenin.

FIG. 2C I illustrates schematically plasmids used for cotransfection assays.

FIG. 2C II shows data on transfection efficiencies measured by counting the GFP positive cells.

FIG. 2D shows quantitative western blot analysis of cyclin D1, c-Myc, p21, cleaved (activated caspase-3, and β-actin as a loading control.

FIG. 3A I shows en face panoramic low-magnification image of methylene blue stained inner surface of dissected intestine (Bar is 5 mm).

FIG. 3A II shows representative bright filed micrographs of hematoxylin and eosin stained cross sections from the intestine.

FIG. 3A III shows immunohistochemical analysis of lesioned-tissues using an antibody specific to activated (cleaved) anti-caspase-3.

FIG. 3B shows the total number of adenomas in both treatment groups.

FIG. 3C shows data on the size distribution bins of lesions.

FIG. 3D shows the total adenoma load across different segments of the GI tract

### DETAILED DESCRIPTION

Germline mutation of the tumor suppressor gene, adenomatous polyosis coli (APC) is responsible for familial adenomatous polyposis (FAP) with nearly 100% risk for colon cancer.

It has been discovered that certain tubulin-binding alkaloids, e.g., noscapine, reduces the dynamics of microtubules, causes a reversible G₂/M arrest in wild type mouse embryonic fibroblasts (MEFs), followed by apoptosis in MEFs isolated from Apc^{Min/+} mice. Treatment of Apc^{Min/+} cells with 9-bromonoscapine restores the regulated expression of β-catenin as judged by decreased expression of reporter gene operation under the control of a TCf-4 response promoter as well as the canonical responsive cell proliferation-inducing cyclin D1 and c-Myc proteins. Both β-catenin levels and activity fell to half the original levels with reduction of cell proliferation-inducing cyclin D1, c-Myc, and induction of cytostatic protein p21 prior to caspase-3 activation. A statistically significant reduction in the number of newly emerging intestinal polyps (to 35% compared with untreated mice) as well as the mean size of polyps (to 42% compared with untreated mice) was shown in Apc^{Min/+} mice treated with a derivative of noscapine. The remaining polys in the mice showed evidence of elevated apoptosis as revealed by immunohistochemistry. There was no evidence of histopathological or hematological toxicity. Thus, in certain embodiments, this disclosure relates to preventing or treating polyposis in FAP patients.

### Adenomatous Polyposis Coli (APC) Gene Mutations

The APC gene is on chromosome 5q21 and consists of 8,535 base pairs organized into 15 exons. Exon 15 contributes 70% to the open reading frame. Hundreds of APC gene mutations are known. The most common germline APC gene mutation involves the introduction of a premature stop codon, either by a frameshift mutation (68%), nonsense mutation (30%), or large deletion (2%), leading to truncation of the protein product in the C-terminal region. The majority of germline and somatic APC mutations occur in exon 15, and more than 50% occur between codons 1286 and 1513—known as the mutation cluster region (MCR). Mutation hotspots are located at codons 1309 and 1061, accounting for approximately 17% and 11% of all germline APC mutations. Wachsmannova-Matelova et al., Neoplasma, 2009, 56(6):486-9, hereby incorporated by reference, identified a common mutation at codon 1309 (3927_{—}3931 delAAAGA) which results in a particularly severe phenotype. In addition to genetic testing for gene mutations, it is contemplated that diagnosis of a mutated APC gene may be by detecting a truncated APC protein or alternate amino acid sequence, and correlating that to a gene mutation.

### Terms

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the terms “prevent” and “preventing” include the prevention of the recurrence, spread or onset. It is not intended that the present invention be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present invention also contemplate treatment that merely reduces symptoms, and/or delays disease progression.

The term “alkyl” refers to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, and the like. The term “substituted alkyl” refers to alkyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally subsituted), alkylester (optionally substituted), arylester (optionally substituted), alkanoyl (optionally substituted), aryol (optionally substituted), and the like.

The term “alkoxy” means an alkyl group linked to oxygen thus: R—O—. In this function, R represents the alkyl group. An example would be the methoxy group CH₃O—. The term “alkenyl” refers to straight or branched chain hydrocarbon groups having 2 to 12 carbon atoms, and at least one double carbon to carbon bond (either cis or trans), such as ethenyl. The term “substituted alkenyl” refers to alkenyl groups substituted with one or more groups, preferably selected from aryl, substituted aryl, heterocyclo, substituted heterocyclo, carbocyclo, substitutes carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), and the like. The term “alkynyl” refers to straight or branched chain hydrocarbon groups having 2 to 12 carbon atoms, and at least one triple carbon to carbon bond, such as ethynyl. The term “substituted alkynyl” refers to alkynyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), and the like.

The terms “cycloalkyl” and “cycloalkenyl” refer to mono-, bi-, or tri homocyclic ring groups of 3 to 15 carbon atoms which are, respectively, fully saturated and partially unsaturated.

The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups may be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), and the like. The terms “carbocyclo”, “carbocyclic” or “carbocyclic group” refer to both cycloalkyl and cycloalkenyl groups. The terms “substituted carbocyclo”, “substituted carbocyclic” or “substituted carbocyclic group” refer to carbocyclo or carbocyclic groups substituted with one or more groups as described in the definition of cycloalkyl and cycloalkenyl.

“Heterocarbocycles” or heterocarbocyclyl” groups are carbocycles which contain from 1 to 4 heteroatoms independently selected from nitrogen, oxygen and sulfur which may be saturated or unsaturated, including ring systems that are not aromatic as a whole, but contain aromatic portions, monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH3).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)2alkyl) such as mesyl and the like, and “Arylsulfonyl” refers to an aryl attached through a sulfonyl bridge (i.e., —S(═O)2aryl).

“Alkylsulfamoyl” refers to an alkyl as defined above with the indicated number of carbon atoms attached through a sulfamoyl bridge (i.e., —NHS(═O)2alkyl), and an “Arylsulfamoyl” refers to an alkyl attached through a sulfamoyl bridge (i.e., (i.e., —NHS(═O)2aryl). “Alkylsulfinyl” refers to an alkyl as defined above with the indicated number of carbon atoms attached through a sulfinyl bridge (i.e. —S(═O)alkyl).

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

The terms “halogen” and “halo” refer to fluorine, chlorine, bromine, and iodine.

The term “aroyl” refers to an aryl group (which may be optionally substituted as described above) linked to a carbonyl group (e.g., —C(O)-aryl).

The terms “including”, “such as”, “for example” and the like are intended to refer to exemplary embodiments and not to limit the scope of the present disclosure.

### Noscopine and Derivatives

Noscapine is 3-(4-methoxy-6-methyl-5,6,7,8-tetrahydro-[1,3]dioxolo[4,5-g]isoquinolin-5-yl)-6,7-dimethoxyisobenzofuran-1(3H)-one and a typical noscapine derivative is 3-(9-halo-4-methoxy-6-methyl-5,6,7,8-tetrahydro-[1,3]dioxolo[4,5-g]isoquinolin-5-yl)-6,7-dimethoxyisobenzofuran-1(3H)-one such as 3-(9-bromoe-4-methoxy-6-methyl-5,6,7,8-tetrahydro-[1,3]dioxolo[4,5-g]isoquinolin-5-yl)-6,7-dimethoxyisobenzofuran-1(3H)-one or 3-(9-chloro-4-methoxy-6-methyl-5,6,7,8-tetrahydro-[1,3]dioxolo[4,5-g]isoquinolin-5 -yl)-6,7-dimethoxyisobenzofuran-1(3H)-one.

In some embodiments, the noscapine derivative is a compound comprising Formula A:

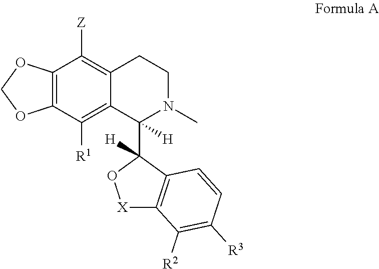

or pharmaceutically acceptable salts, prodrugs or derivatives thereof.

wherein Z is a halogen, nitro, or nitrogen wherein nitrogen may be optionally substituted with R⁴;

X is carbonyl (C═O) or methylene (CH₂) optionally substituted with R⁴;

R¹, R², and R³ are each independently an alkoxy optionally substituted with one or more R⁴; R⁴ is independently selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with R⁵;

R⁵ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl. In a typical embodiment, R¹, R², and R³ are each methoxy.

In certain embodiments, Z is hydrogen, X is methylene (CH₂) or a carbonyl (C═O) group optionally substituted with R⁴; R¹, R², and R³ are each independently an alkoxy optionally substituted with one or more R⁴.

To the extent that the disclosed compounds, and salts thereof, may exist in their tautomeric form, all such tautomeric forms are contemplated herein as part of the present disclosure.

The compounds can be in a free base form or in a salt form (e.g., as pharmaceutically acceptable salts). Examples of suitable pharmaceutically acceptable salts include inorganic acid addition salts such as sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with an acidic amino acid such as aspartate and glutamate; alkali metal salts such as sodium and potassium; alkaline earth metal salts such as magnesium and calcium; ammonium salt; organic basic salts such as trimethylamine, triethylamine, pyridine, picoline, dicyclohexylamine, and N5N′-dibenzylethylenediamine; and salts with a basic amino acid such as lysine and arginine. The salts can be in some cases hydrates or ethanol solvates. The stoichiometry of the salt will vary with the nature of the components. Representative compounds include

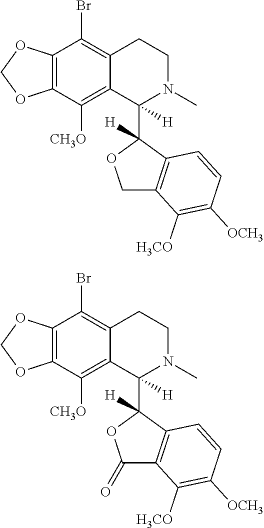

The compounds described herein may be administered in the form of prodrugs. By “prodrug” is meant, for example, any compound (whether itself active or inactive) that is converted chemically in vivo into a biologically active compound as described herein following administration of the prodrug to a subject.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds according to formula A.

The compounds can be prepared by performing electrophilic aromatic substitution on the isoquinoline ring of noscapine, typically under conditions that do not result in significant hydrolysis of the noscapine framework. The substituents typically are added to the 9-position on the isoquinoline ring, although yields can be optimized and by-products may be present and need to be removed during a purification step. More optimized syntheses of representative compounds, such as 9-bromo-nos and Red-9-bromo-nos, are provided below.

Briefly, the nitration of the isoquinoline ring in noscapine can be accomplished by using stoichiometric silver nitrate and a slight excess of trifluoroacetic anhydride.

The halogenation of noscapine involved various procedures, which varied depending on the particular halogen, as summarized below in Scheme 1.

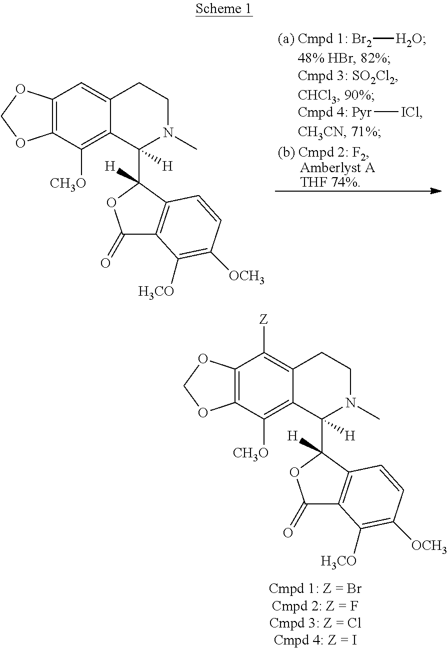

Noscapine can be brominated at the 9-position by reacting noscapine with concentrated hydrobromic acid. Noscapine can be fluorinated using the fluoride form of Amberlyst-A 26, or by Br/F exchange. Iodination of noscapine typically required low-acid conditions. One successful approach for preparing 9-Iodo-nos involved treating a solution of noscapine in acetonitrile with pyridine-iodine chloride at room temperature for 6 hours followed by raising the temperature to 100° C. for another 6 hours.

For those skilled in the art, incorporation of other substituents onto the 9-position of the isoquinoline ring, and other positions in the noscapine framework, can be readily realized. Such substituents can provide useful properties in and of themselves or serve as a handle for further synthetic elaboration.

Noscapine analogs may be synthesized according to previously reported methods. [Zhou et al. (2003) Mol Pharmacol 63: 799-807 and Aneja et al. (2006) Biochem Pharmacol 72:415-426].

One prepares (S)-6,7-Dimethoxy-3-((R)-4-methoxy-6-methyl-9-nitro-5,6,7,8-tetrahydro-[1,3]dioxolo[4,5-g]iso-quinolin-5-yl)isobenzofuran-1(3H)-one (9-nitro-nos) by the aromatic nitration of (S)-6,7-dimethoxy-3-((R)-4-methoxy-6-methyl-5,6,7,8-tetrahydro-[1,3]dioxolo[4,5-g]isoquinolin-5-yl)isobenzo-furan-1(3H)-one (noscapine) using silver nitrate in acetonitrile and TFAA at 25° C. in accordance with the procedures disclosed in WO2008/109609.

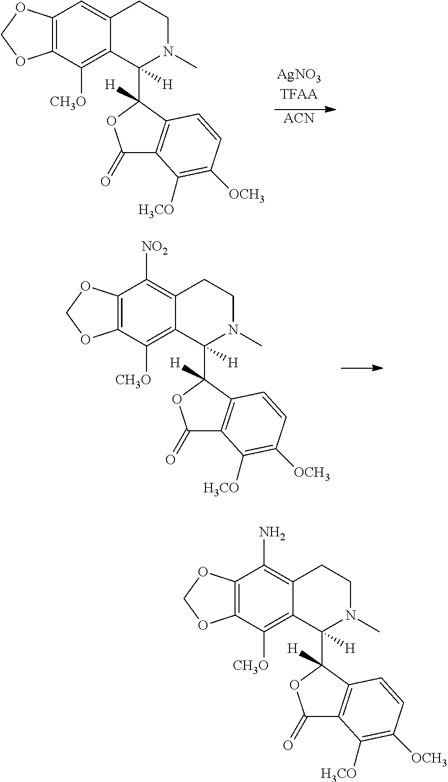

Reduction of the nitro group to an amine allows for amino acid couplings or transformation to other derivative such as alkyl amides, secondary and tertiary amines, etc.

### Formulations

Pharmaceutical compositions disclosed herein can be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure can also form internal salts, and such compounds are within the scope of the disclosure. When a compound contains a hydrogen-donating heteroatom (e.g. NH), salts are contemplated to cover isomers formed by transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Examples of structuring a compound as prodrugs can be found in the book of Testa and Caner, Hydrolysis in Drug and Prodrug Metabolism, Wiley (2006) hereby incorporated by reference. Typical prodrugs form the active metabolite by transformation of the prodrug by hydrolytic enzymes, the hydrolysis of amides, lactams, peptides, carboxylic acid esters, epoxides or the cleavage of esters of inorganic acids.

Pharmaceutical compositions typically comprise an effective amount of a compound and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet (2006) 21(3):173-85, entitled “Human carboxylesterase isozymes: catalytic properties and rational drug design.”

Generally, for pharmaceutical use, the compounds can be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount,” by which it is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which can be administered as a single daily dose, divided over one or more daily doses. The amount(s) to be administered, the route of administration and the further treatment regimen can be determined by the treating clinician, depending on factors such as the age, gender and general condition of the patient and the nature and severity of the disease/symptoms to be treated. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which can include plasticizers, pigments, colorants, stabilizing agents, and glidants. Delayed release, extended release, and/or pulsatile release dosage formulations can be prepared as described in standard references such as “Pharmaceutical dosage form tablets,” eds. Liberman et. al. (New York, Marcel Dekker, Inc., 1989), “Remington—The science and practice of pharmacy,” 20th ed., Lippincott Williams & Wilkins, Baltimore, Md., 2000, and “Pharmaceutical dosage forms and drug delivery systems,” 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material can contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants can be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles can also contain minor amount of nontoxic auxiliary substances such as wetting or emulsifying agents, dyes, pH buffering agents, or preservatives.

The compositions described herein can be formulation for modified or controlled release. Examples of controlled release dosage forms include extended release dosage forms, delayed release dosage forms, pulsatile release dosage forms, and combinations thereof

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit ® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS can be mixed together in any desired ratio in order to ultimately obtain a sustained-release formulation having a desirable dissolution profile. Desirable sustained-release multiparticulate systems can be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers can also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray- congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, each dosage unit in the capsule can comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that can or can not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles can be formulated to provide immediate release, beads and granules are generally employed to provide delayed release.

### Combination Therapies

The cancer treatments disclosed herein can be applied as a sole therapy or can involve, conventional surgery or radiotherapy or chemotherapy. Such chemotherapy can include one or more of the following categories of anti-tumor agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-Her2 antibody trastuzumab and the anti- epidermal growth factor receptor (EGFR) antibody, cetuximab), farnesyl transferase inhibitors, tyrosine kinase inhibitors and serine/threonine kinase inhibitors, for example inhibitors of the epidermal growth factor family for example EGFR family tyrosine kinase inhibitors such as: N-(3-chloro-4-fluorophenyl)-7-methoxy-6-(3-morpholinopropoxy)quinazolin-4-a mine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors of phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvβ3 function and angiostatin);

(vi) vascular damaging agents such as Combretastatin A4;

(vii) antisense therapies, for example those which are directed to the targets listed above, such as an anti-RAS antisense; and

(viii) immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies, and approaches using the immunomodulatory drugs thalidomide and lenalidomide [Revlimid®].

### Experimental

### EXAMPLE 1

### 9-Bromonoscapine Induced Cell Death of Ape^{Min/+} Murine Embryonic Fibroblasts (MEFs)

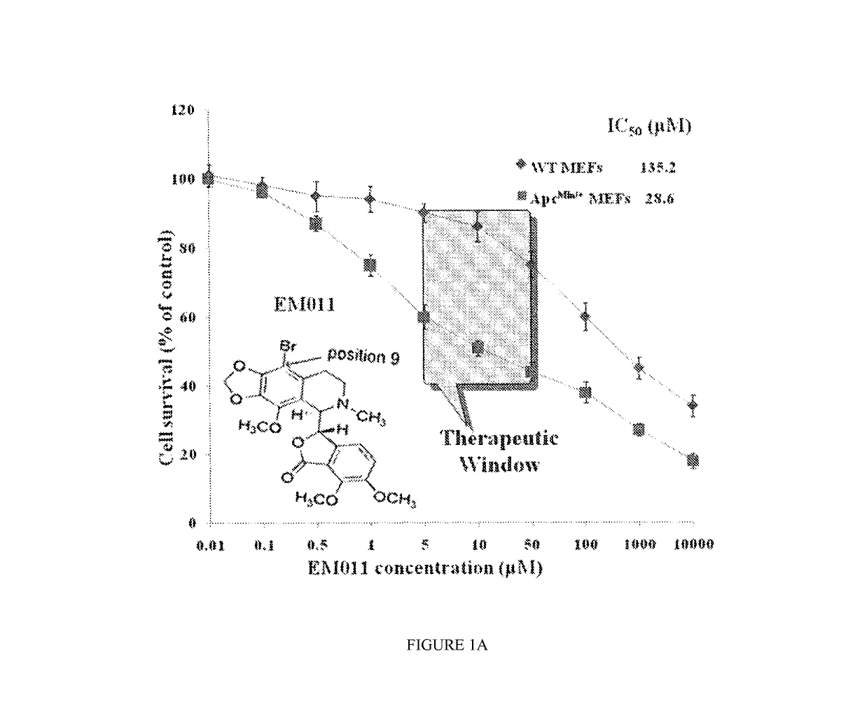

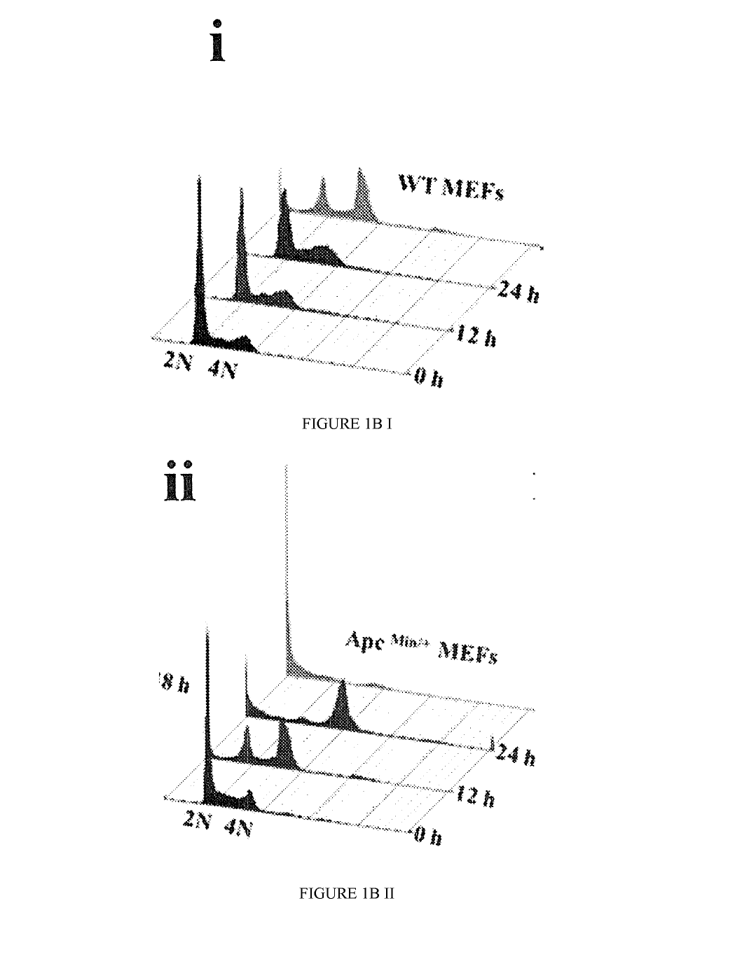

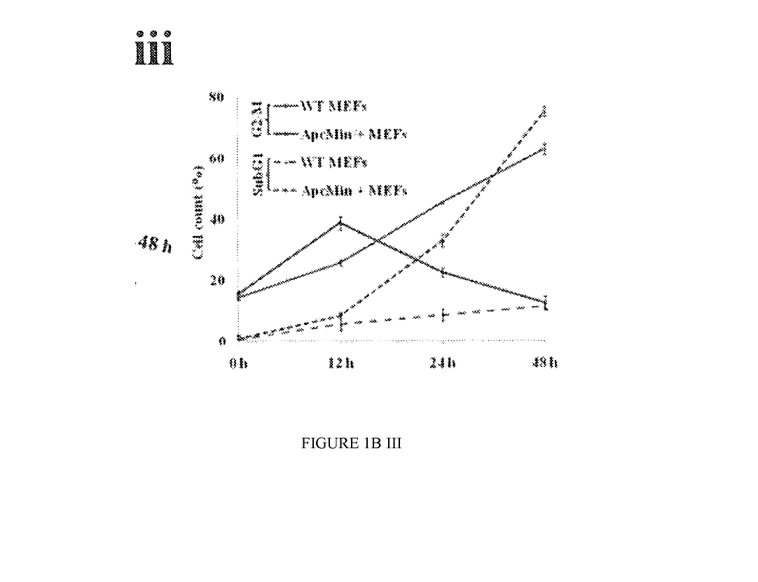

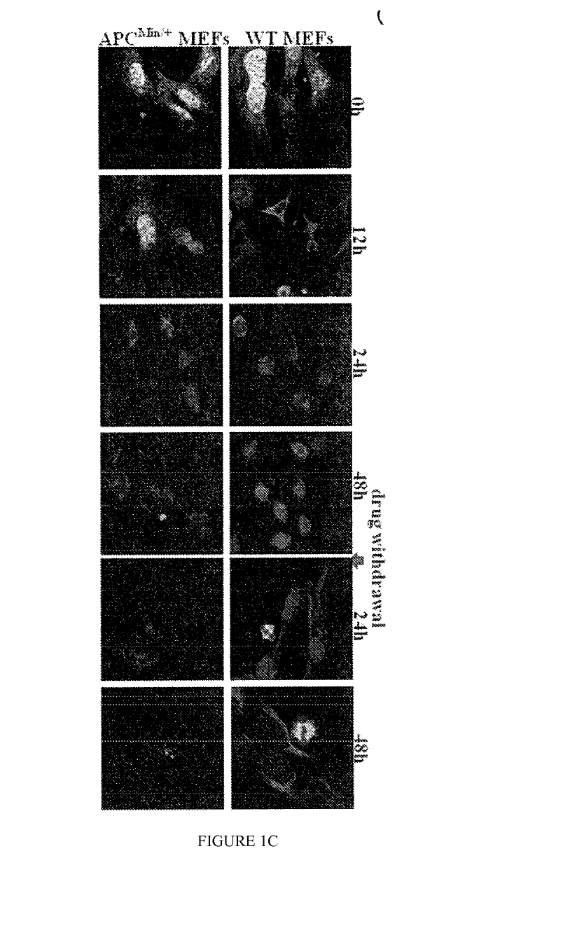

Murine embryonic fibroblasts (MEFs) isolated from Apc^{Min/+} mice and wild type mice were treated with either vehicle solution of DMSO or varying concentrations of 9-bromonoscapine for 48 hours. Viable and dead cells were counted in triplicate using exclusion of the vital dye, trypan blue. As shown in FIG. 1A, the IC₅₀ curve of WT MEFs was clearly different than that of Apc^{Min/+} MEFs. They revealed a marked difference in their susceptibility to 9-bromonoscapine in that these cells were 4.7 times more sensitive with a steeper death-curve than WT MEFs (IC₅₀=135.2 μM for WT MEFs v/s IC₅₀=28.6 μM for Apc^{Min/+} MEFs). This provided a therapeutic window (5-50 μM) for selective killing of Apc^{Min/+} MEFs while causing minimal damage to the WT MEFs (FIG. 1A).

Fluorescent-activated cell sorting (FACS) analysis of DNA content showed a decline in G1 and a rise in G2/M of both WT MEFs (FIG. 1B i, iii) and Apc^{Min/+} MEFs (FIG. 1B ii, iii). In contrast to the WT MEFs, Apc^{Min/+} MEFs showed a distinct shift of cells to a sub-G1 DNA content of <2N at 48 hours of treatment suggesting DNA-degradation associated with apoptosis. Confocal microscopic examination of the MT arrays and chromosomes in treated cells reveals a typical mitotic arrest with bipolar prometaphase spindles with tightly condensed chromosomes in WT MEFs that was reversed as the cells resumed normal mitosis as early as 24 hours after withdrawal of treatment. (FIG. 1C, upper panels). In contrast, highly disorganized, often multipolar mitoses were visible with less condensed chromosomes at 48 hours of treatment in Apc^{Min/+} MEFs, which did not recover after the drug removal but rather appeared to die 48-hours post treatment (FIG. 1C, lower panels).

### EXAMPLE 2

### 9-Bromonoscapine Causes Apoptotic Onset

Apoptotic cells that externalize the normal internal membrane lipid, phosphatidylserine, were analyzed by binding a fluorescently conjugated PS-binding protein, Annein V-Alexa Fluor488. These cells can be distinguished from the late apoptotic cells that also allow the DNA dye, Propidium iodide (PI) to penetrate the cell membranes allowing intracellular DNA to bind it. As shown in FIG. 1D i-ii, 73% of Apc^{Min/+} MEFs were in early and late stages of apoptosis after a 48 hour treatment with 9-bromonoscapine (50 μM) as compared to a modest 21.6% WT MEFs under identical treatment regimen (FIG. 1D i-ii). 9-Bromonoscapine causes a selective cell death (apoptosis) in MEFs isolated from Apc^{Min/+} mice when compared to the WT MEFs providing a therapeutic window from 5-50 μM range.

### EXAMPLE 3

### 9-Bromonoscapine Effects β-Caternin Activity

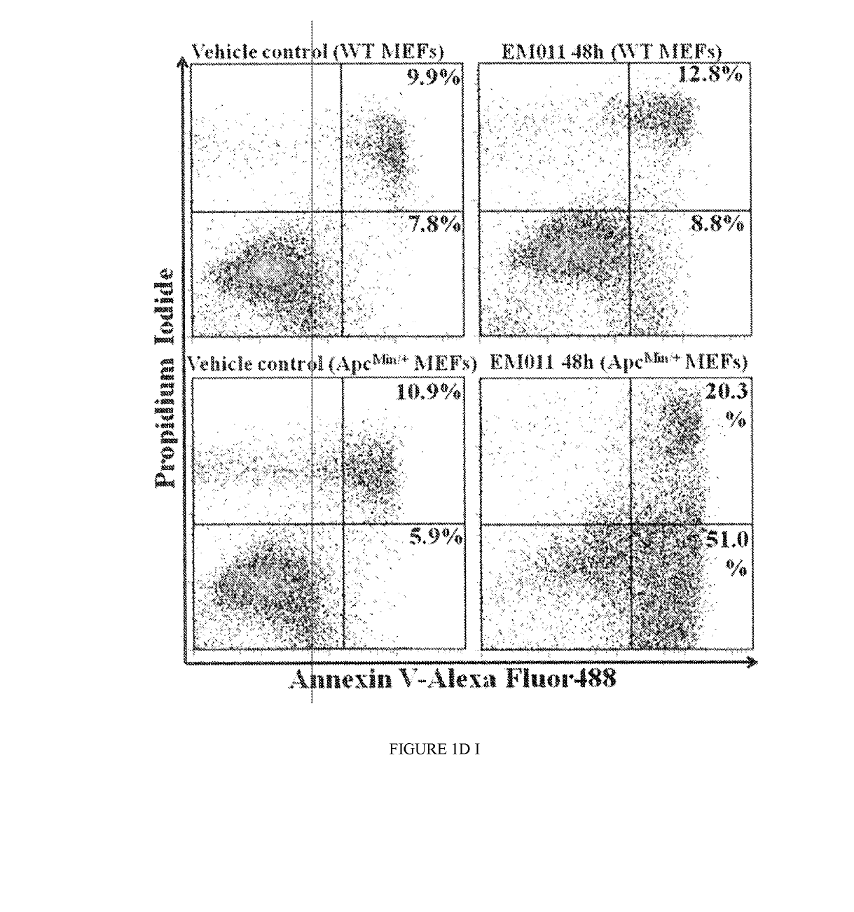

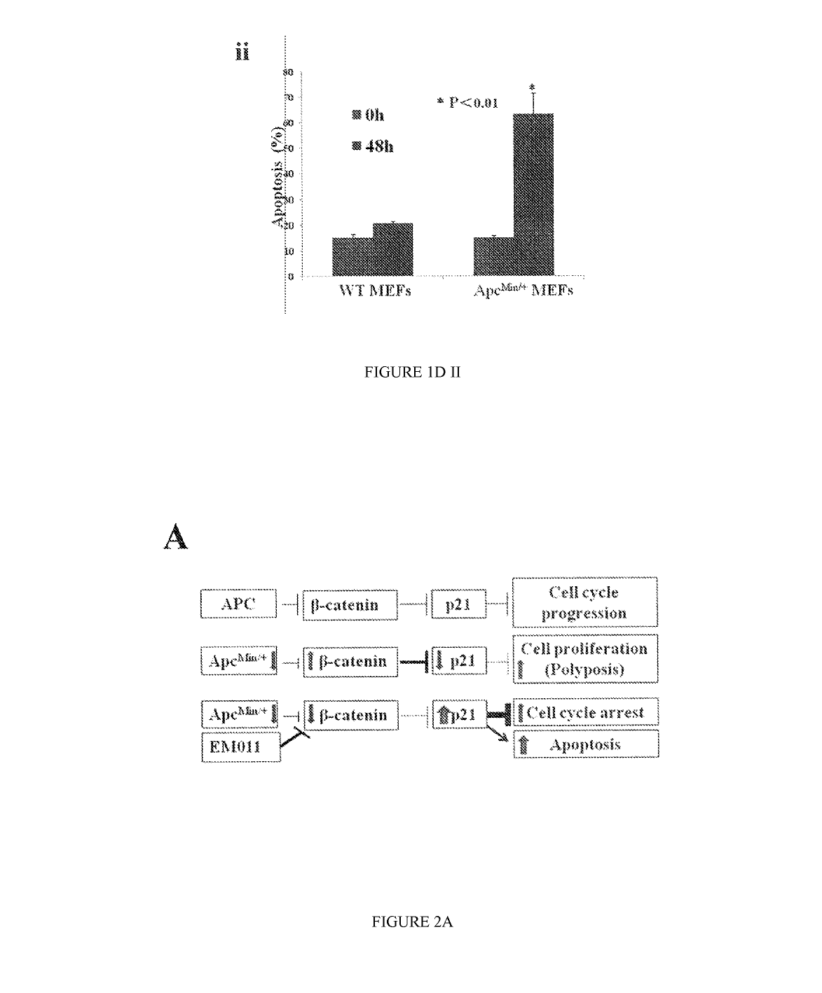

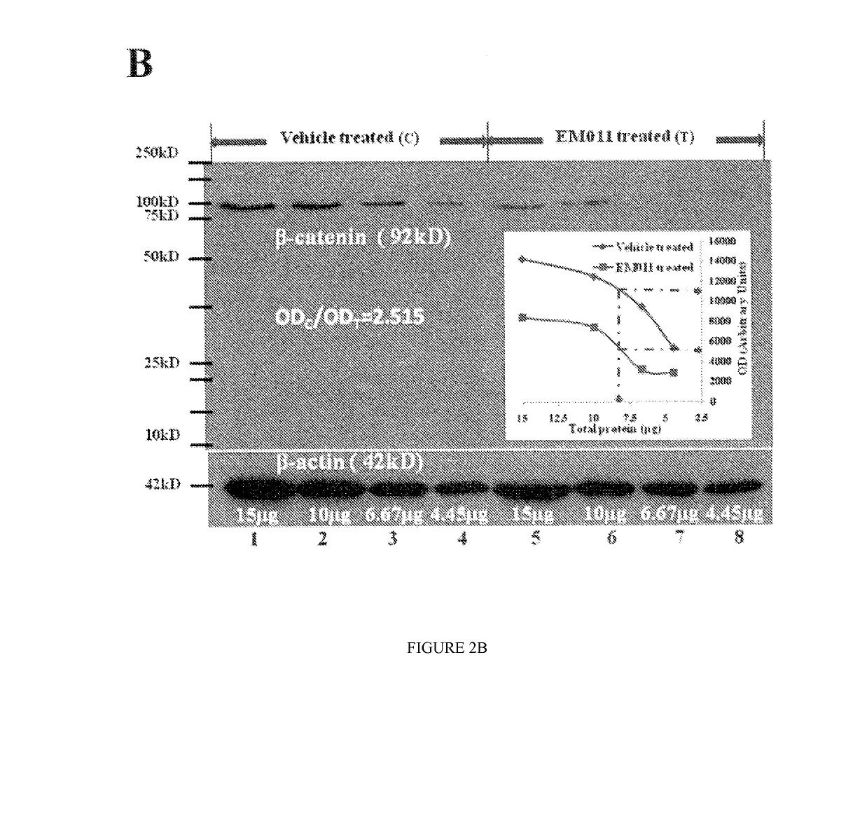

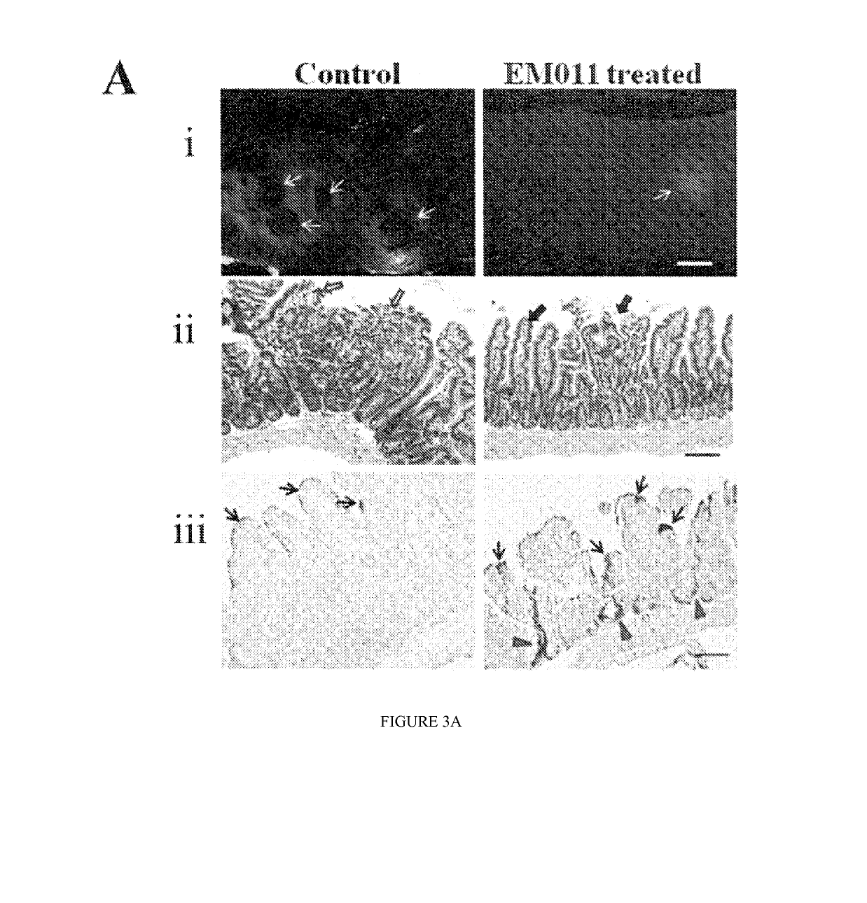

HCT116 cells harbor an in-frame deletion of one phosphorylation site (Ser⁴⁵) in β-catenin that renders is partially active but has wild type APC. As shown in FIG. 2C iii, these cells show a 31% decline in the β-calenin activity as early as 8 hours after treatment with 9-bromonoscapine. In cell lines that are deficient in APC function either due to mutational inactivation such as in DLD1 cells or due to a heterozygous deletion as in Apc^{Min/+} MEFs, the β-catenin activity was significantly lowered (p<0.01) to 48% and 59% respectively (FIG. 2C iii). These results are consistent with the hypothesis that increasing the cytoplasmic abundance of MT plus ends by treatment with 9-bromonoscapine restores the appropriate up-regulation of β-catenin levels and activity. Because β-catenin is a negative regulator of CDKs inhibitor p21, p21 levels will rise upon restored downregulation of β-catenin and the cell cycle progression will be inhibited allowing cellular apoptosis (FIG. 2A). The rise in p21 in colorectal carcinoma cells (HCT116) has been shown to be associated with the induction of apoptosis. Hohla et al., Cell Cycle, 2009, 8(19):3149-3156.

A two-fold decrease in β-catenin levels follow the treatment, of Apc^{Min/+} MEFs by 9-bromonoscapine (FIG. 2B). This decrease in β-catenin levels does translate in reduction of its activity. A reporter gene luciferase under the control of three cloned copies of a conserved Tcf4/β-catenin responsive element was used and compared with an internal control, i.e., another reporter Renilla activity driven by a (β-catenin independent) promoter (CMV) in all three cell types tested (HCT116, DLD, Apc^{Min/+} MEFs). All three cell types chosen showed adequate transfection efficiencies (tested independently by co-transfection of a GFP expression construct in Apc^{Min/+} MEF, FIG. 2C i-iii).

Positively responsive target genes cyclin-D l and c-Myc, and the negatively responsive gene, p21, respond with the restoration of partial loss of function by depleted APC activity after 9-bromonoscapine treatment. FIG. 2D shows that the cyclin D1 and c-Myc proteins were down while p21 levels were elevated in response to 9-bromonoscapine treatments. Furthermore, caspase-3 was activated as measured by the rising levels of cleaved active caspase-3. Taken together, all of these data are in line with the cellular observation of 9-bromonoscapine treatment of cells in FIG. 1, i.e., the Apc^{Min/+} cells were driven to apoptosis by 9-bromonoscapine treatment more efficiently than the wild type cells.

### EXAMPLE 4

### 9-Bromonoscapine Prevents Growth of Polyps and the Formation of Polys in Apc^{Min/+} Mice

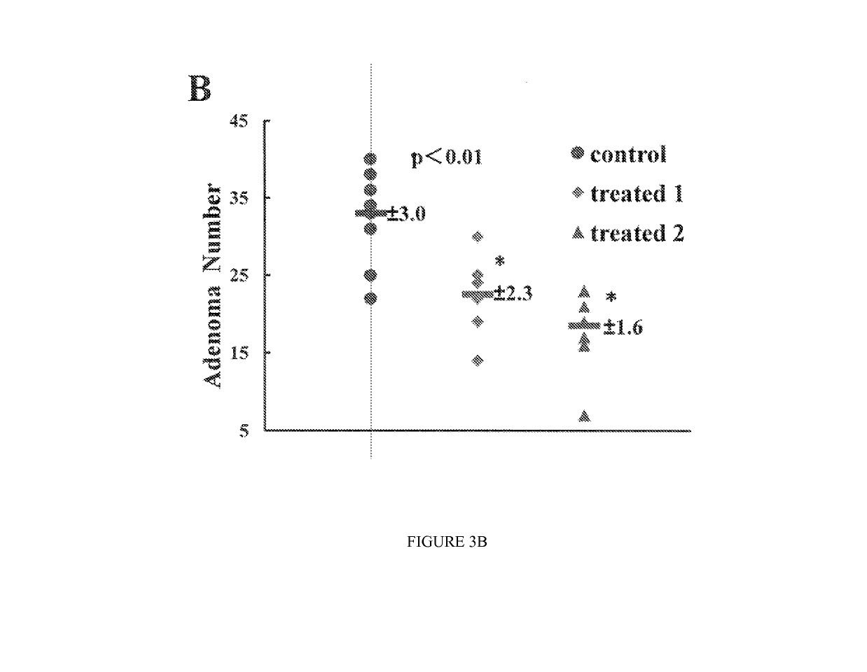

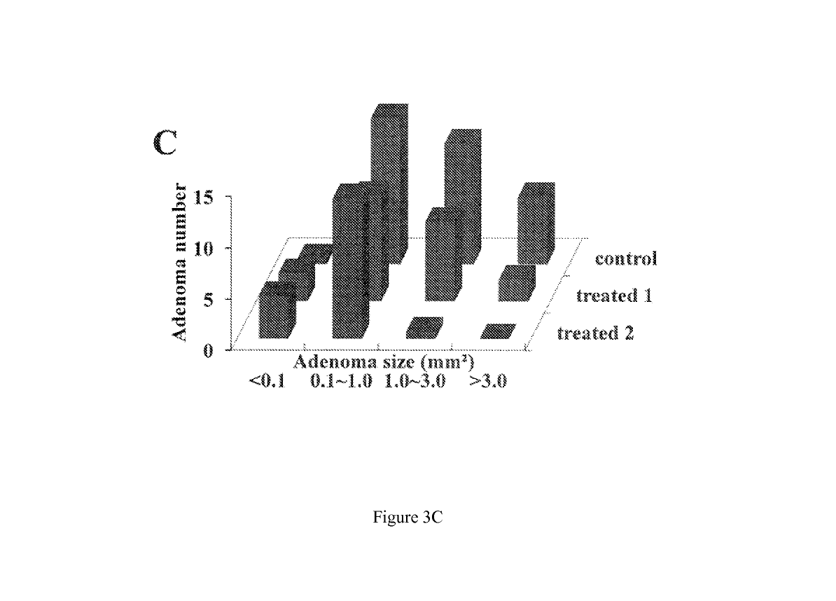

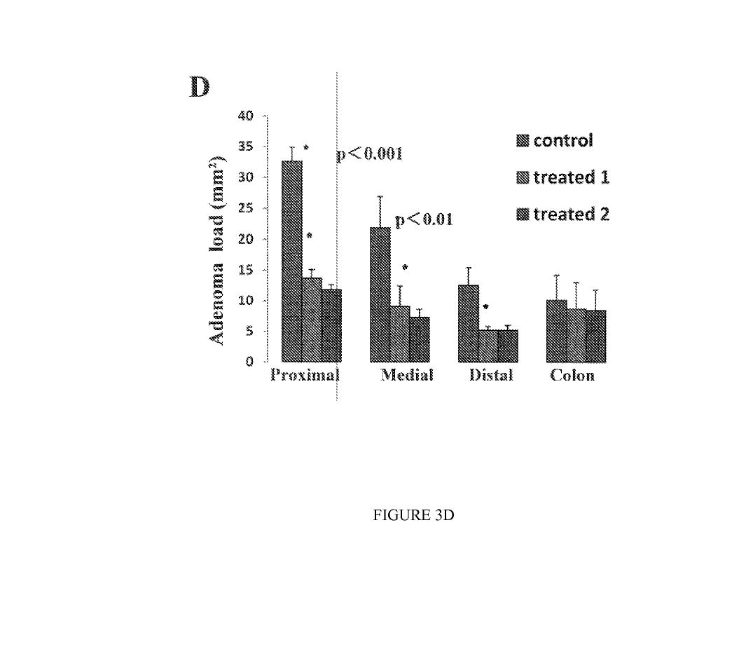

Mice were treated either with the vehicle solution alone (control) or 150 mg/kg body weight of 9-bromonoscapine. The mice were analyzed for the number of polyps throughout the gastrointestinal tract of animals. FIG. 3A i shows the en face panoramic image of methylene blue stained inner surface of a dissected intestine. The pronounced decrease due to 9-bromonoscapine treatment both in the number and overall areas of intestinal lesions are readily visible. Representative bright filed micrographs of hematoxylin and eosine stained 5 μm cross sections from the intestine of vehicle treated control group (FIG. 3A ii, left panel) and 9-bromonoscapine treated group (FIG. 3A ii, right panel) show hyperplastic tissue lesions. In 9-bromonoscapine treated animals, the lesions were not clustered and, when found, seem much more restricted to the individual microvilli and were subdued in appearance (FIG. 3A ii, right panel). Using a cleaved (active) anti-caspase 3 antibody, the immunohistochemical analysis of these lesioned-tissues revealed the normal apoptotic zones along the apices of intestinal villus in both the untreated control and 9-bromonoscapine treated animals (FIG. 3A iii). In sharp contrast to the untreated control aminals, the 9-bromonoscapine treated animals showed additional copious apoptotic foci abnormally in the basal areas of the villus (FIG. 3A iii).

To distinguish between neopolyposis post-treatment versus the growth of apparently existing polyps, two treatment regimens were followed. Treated 1: 9-bromonoscapine oral treatment (150 mg/kg body weight) began after 8 weeks of age and continued daily for 12 weeks prior to the experimental end point (in 20 weeks): Treated 2: 9-bromonoscapine oral administration began 21 days after birth at the same dose level for time matched period until the experimental end point. At this time, animals were euthanized and the entire GI tract was dissected out and flushed with PBS. The intestine was then removed from the colon and cut into three equal segments: proximal, medial, and distal. The colon and all three segments of the intestine were cut open longitudinally, stained with methylene blue prior to examination under a dissecting microscope fitted with a micrometer to aid the measurements. The vertex and co-vertex of each elliptical lesion was measured. The area was then calculated by multiplying the vertex, co-vertex and π (3.14). The total number of adenomas showed a significant decline in both treatment groups as compared with the sham-treated controls (FIG. 3B). The size distribution bins of lesions are displayed along the abscissa (X-axis) and the numbers of adenomas along the ordinate (Y-axis). Most notable features are significant decreases in the big lesions (>3 mm² and >1 mm²), which remained small, and show up as an apparent concomitant increase in small bin (<0.1 mm²) (FIG. 3C). The statistical analysis of variance revealed highly significant prevention of the adenoma load across the proximal intestine from 32.2±2.3 mm² to 13.7±1.4 mm² (Treated 1) and 11.8±0.8 mm² (Treated 2)(p<0.01) and distal intestine from 12.5±2.9 mm² to 5.2±0.6 mm2 (Treated 1) and 5.2±0.8 mm2 (Treated 2)(p<0.01) (FIG. 3D). There was no visible difference between the groups of treated 1 and treated 2. The colonic adenomas are known to be rare in Apc^{Min/+} mouse models. The occasional adenoma within the colon did not reveal any apparent differences (FIG. 3D).

### EXAMPLE 5

### Toxicology of 9-Bromonoscapine

Hematoxylin and eosin stained 5 μm sections of paraffin-embedded brain, lung, liver, kidney, thymus, spleen, heart, and sciatic nerve tissues were analyzed. Blinded observations did not reveal any significant differences in the tissue architecture. Organ associated toxicity was assessed by measuring organ functions in vehicle-treated and 9-bromonoscapine treated groups. Liver function tests (total bilirubin, alanine transaminase, aspartate aminotransferase, and alkaline phosphate levels) and renal functions tests (blood urea nitrogen and creatinine levels) were similar between drug treated and vehicle-treated groups. Systemic homeostasis (albumin, total protein, glucose) and electrolyte balances (Na⁺, K⁺, TCO₂) also showed not distinguishable profiles among the three groups. There were no significant differences in WBC, RBC counts, hemoglobin concentration, hematocrit, and platelet counts.

1. A method of treating or preventing cancer comprising administering a pharmaceutical composition comprising a noscapine derivative to a subject diagnosed with a mutated adenomatous polyposis coli (APC) gene.

2. The method of claim 1, wherein the noscapine derivative is a compound comprising Formula A: 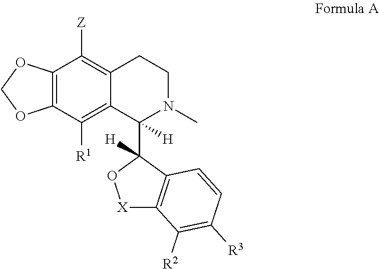
or pharmaceutically acceptable salts, esters, or prodrugs thereof.
wherein Z is a halogen, nitro, or nitrogen wherein nitrogen may be optionally substituted with R⁴; X is methylene (CH₂) optionally substituted with R⁴; R¹, R², and R³ are each independently an alkoxy optionally substituted with one or more R⁴; R⁴ is independently selected from alkyl, alkenyl, alkanoyl, halogen, nitro, cyano, hydroxy, amino, mercapto, formyl, carboxy, carbamoyl, alkoxy, alkylthio, alkylamino, dialkylamino, alkylsulfinyl, alkylsulfonyl, arylsulfonyl, carbocyclyl, aryl, and heterocyclyl wherein R⁴ is optionally substituted with R⁵;
R⁵ is selected from halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

3. The method of claim 2, wherein R¹, R², and R³ are each alkoxy.

4. The method of claim 1, wherein the subject is not diagnosed with colon cancer.

5. The method of claim 4, wherein the subject is at risk of developing colon cancer.

6. The method of claim 1, herein the subject is diagnosed with colonic polyps.

7. The method of claim 1, wherein the pharmaceutical composition is administered in combination with a non-steroidal anti-inflammatory agent.

8. The method of claim 1, wherein the pharmaceutical composition is administered in combination with aspirin or sulindac.

9. The method of claim 1, wherein the pharmaceutical composition is administered in combination with a second chemotherapeutic agent.

10. The method of claim 9, wherein the chemotherapeutic agent is wherein the second chemotherapeutic agent is docetaxel, cis-platin, 5-fluorouracil, tegafur-uracil, capecitabine, leucovorin, oxaliplatin, irinotecan, panitumumab, oblimersen, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib, anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, gefitinib, erlotinib, dasatinib, imatinib, bevacizumab, combretastatin, thalidomide, and lenalidomide.

11. The method of claim 1, wherein the pharmaceutical composition is administered in combination with cimetidine.

12. The method of claim 1, wherein the pharmaceutical composition is administered in combination with a cancer vaccine.

13. The method of claim 1, wherein the pharmaceutical composition is administered in combination with a tumor-associated antigen, 5T4, with a pox virus vector.

14. The method of claim 1, wherein the pharmaceutical composition is administered in combination with vitamin B6 or calcium.

15. The method of claim 1, wherein the subject is diagnosed with a tumor confined within the wall of the colon.

16. The method of claim 1, wherein the pharmaceutical composition is administration before or after the subject undergoes surgical removal of the colon.

17. The method of claim 1, wherein the pharmaceutical composition is administration before or after the subject undergoes radiation therapy.
